(19) United States
(12) Patent Application Publication
(10) Pub. No.: US 2024/0009246 A1
(43) Pub. Date: Jan. 11, 2024

(54) CELLULAR-ANTIMICROBIAL COMBINATION COMPOSITION AND METHODS FOR TREATMENT OF BACTERIAL INFECTIONS OF JOINTS AND SOFT TISSUES

(71) Applicant: COLORADO STATE UNIVERSITY RESEARCH FOUNDATION, Fort Collins, CO (US)

(72) Inventors: Steven Dow, Fort Collins, CO (US); Lynn Pezzanite, Fort Collins, CO (US); Laurie R. Goodrich, Bellvue, CO (US)

(21) Appl. No.: 18/220,689

(22) Filed: Jul. 11, 2023

Related U.S. Application Data
(63) Continuation of application No. PCT/US2022/011627, filed on Jan. 7, 2022.
(60) Provisional application No. 63/136,468, filed on Jan. 12, 2021.

(51) Int. Cl. A61K 35/28; A61K 38/14; A61K 31/43; A61K 31/7056; A61K 31/407; A61K 31/546; A61P 31/04

(52) U.S. Cl. CPC A61K 35/28; A61K 38/14; A61K 31/43; A61K 31/7056; A61K 31/407; A61K 31/546; A61P 31/04

(57) ABSTRACT

Embodiments of the present invention generally relate to compositions and methods for treating acute or chronic bacterial infections of joints, tendons, ligaments, implants and associated soft tissues. In other embodiments, compositions of use herein generally relate to activated MSCs or iMSCs combined with antibiotics having reduced cytotoxicity for the treatment of bacterial infections of joints, tendons, ligaments, implants and associated soft tissue structures.

This application is a U.S. Continuation Application of International Application PCT/US2022/011627, filed Jan. 7, 2022, which claims priority to U.S. Provisional Application No. 63/136,468, filed Jan. 12, 2021. These applications are incorporated herein by reference in their entirety for all purposes.

### FIELD

Embodiments of the present invention generally relate to compositions and methods for treating acute and/or chronic infections of joints, tendons and ligaments and related soft tissues of a subject. In other embodiments, compositions of use herein generally relate to compositions including activated mesenchymal stromal cells (MSCs) or activated MSC derived from induced pluripotent stem cells (iPSCs) (iMSCs) and combined directly or indirectly with bactericidal and non-cytotoxic antibiotics. In other embodiments, compositions disclosed herein can be used for the treatment of acute and/or chronic bacterial infections of implants and associated soft tissues thereof.

### BACKGROUND

Antimicrobial resistance represents a major threat to public health. In orthopedic and trauma surgery, postoperative infection occurs in close to 30% of cases, with treatment success rates varying from about 57% to about 88% depending on many factors, including for example, whether bacterial biofilms have formed. Infections with multi-drug resistant bacterial strains negatively impact clinical outcomes and increase healthcare expenditures. Formation of bacterial biofilms in chronic orthopedic infections is also a major barrier to successful treatment with antibiotics alone, even when apparent drug resistance has not developed. Joint infections are typically treated with joint lavage (either closed or open lavage), combined with long-term antibiotic therapy, administered by the iv or oral routes. Tendon and ligament infections are typically managed by drainage of the infected tendon sheath, followed by long-term antibiotic therapy. Prosthetic joint infections (PJI) with biofilm formation are typically managed by surgical, open lavage and debridement, and in many cases by removal of the infected prosthesis, followed by long-term antibiotic therapy.

In addition, the rapid developmental formation of bacterial biofilms and acquisition of drug resistance necessitates advancement of novel strategies to combat these chronic and debilitating infections. Further, due to an increase globally in antibiotic resistant infections and multi-drug resistant infections, there remains a critical unmet need for additional therapies to be combined with conventional antibiotic therapy for the treatment of acute and chronic orthopedic and soft tissue infections.

### SUMMARY

Embodiments of the present invention generally relate to compositions and methods for treating acute and/or chronic infections of joints, tendons and ligaments and related soft tissues of a subject. In other embodiments, compositions of use herein generally relate to compositions including activated mesenchymal stromal cells (MSCs) or activated MSC derived from induced pluripotent stem cells (iPSCs) (iMSCs) and combined directly or indirectly with bactericidal and non-cytotoxic antibiotics. In other embodiments, compositions disclosed herein can be used for the treatment of acute and chronic bacterial infections of joints, tendons, ligaments and/or their associated soft tissues. In certain embodiments, these conditions can include acute or chronic microbial (e.g. bacterial) infections of joints, tendons, or ligaments or other musculoskeletal soft tissues, whether occurring spontaneously, due to trauma, surgery, or other injury. In some embodiments, compositions disclosed herein concern combinations of TLR ligand or agonist activated MSCs or iMSCs; in combination with bactericidal and non-cytotoxic antibiotics having activity against infection caused by bacteria and related organisms, including Gram negative (Gram−) and Gram positive (Gram+) bacteria and mycobacteria and mycoplasmas. In accordance with these embodiments, these combinations can be used in the treatment of infections of joints, tendons, ligaments, associated soft tissues, bone abscess or prosthetic implants or catheters of a subject.

In certain embodiments, microbial infections can include, but are not limited to, drug-resistant strains of bacteria (e.g. MRSA strains). In other embodiments, the infecting bacteria have formed biofilms, either within joints or tendon sheaths, or on orthopedic implants (e.g., knee and hip joint replacements). In some embodiments, intra-articular (IA) administration of a TLR3 ligand activated MSCs or activated iMSCs alone or in combination with anti-microbial therapies to joints or implants of a subject are contemplated herein. In other embodiments, compositions and methods disclosed herein concern treating a subject with an infection of joints, tendons or ligaments or associated soft tissues, for example, infections of prosthetic implants, and infections of the prosthetic implants with associated bacterial biofilms. In certain embodiments, treatments disclosed herein can be used to reduce onset of or prevent a microbial infection.

In other embodiments, localized treatment to reduce, prevent or treat infections of joints, tendons, ligaments, or associated soft tissues by direct injection into joints, tendons, or associated soft tissues, with a composition including, but not limited to, activated MSC, combined directly or indirectly with bactericidal and non-cytotoxic antibiotics, for a single combined injection into infected sites is contemplated herein. In certain embodiments, the injections of these composition can be repeated at pre-determined intervals for multiple injections and prolonged treatment where needed. In other embodiments, treatments using compositions disclosed herein can include treatment of infections of chronic indwelling catheters (e.g., PIC lines, bladder catheters) by direct infusion of activated MSCs (e.g. TLR activated MSCs or iPSCs) separately or in combination with bactericidal and non-cytotoxic antibiotics into the infected catheter to treat the infection.

In some embodiments, TLR3 ligands can be used to activate MSCs or iMSCs for use in compositions directly administered into the infected areas including joints, tendon or ligament or associated soft tissues of a subject. In accordance with these embodiments, TLR3 ligands of use to activate MSCs or iMSCs can include, but are not limited to, one or more of polyadenylic-polyuridylic acid (poly(A:U), polyinosine-polycytidylic acid (pIC), and UV- or otherwise inactivated viral particles (e.g., poxvirus particles or other DNA viral particles). In certain embodiments, activation of MSCs or iMSCs can include exposure to one or more TLR3 ligand for about 30 minutes to less than 12 hours.

In some embodiments, intra-articular (IA) administration of activated MSCs or activated iMSCs alone or in combination with one or more anti-microbial therapies to joints or prosthetic implants or directly applied to an infected region in a subject are contemplated herein. In other embodiments, direct injection of activated MSCs or iMSCs in fixed combination with bactericidal and/or non-cytotoxic antibiotics or other antimicrobial, non-cytotoxic compounds (e.g., antimicrobial peptides) into infected joints, tendons, ligaments, or associated soft tissues are contemplated. In accordance with these embodiments, local injection of compositions or combination compositions disclosed herein can be administered to a subject in need thereof by syringe and needle injection, by implanted catheter or other local delivery device. In some embodiments, treatment intervals can be every other day, twice weekly to once every three months or other pre-determined regimen.

In certain embodiments, antimicrobial agents of use in compositions disclosed herein can include bactericidal antibiotics or other anti-bacterial compounds (e.g., antimicrobial peptides) that have been determined to have minimal cellular cytotoxicity (for example, towards MSCs such as activated MSC, activated iMSCs or to surrounding joint or tendon or ligament tissues). In accordance with these embodiments, antibiotics and/or other antimicrobial factors (e.g. antimicrobial peptides) can be injected directly together with the activated MSC or iMSC for maximal benefit. In some embodiments, an antimicrobial peptide can include, but are not limited to, LL-37, beta-defensin, CXCL10, surfactant A or the like. In some embodiments, antibiotics for treatment of Gram+ infections, based on bactericidal activity and minimal cellular cytotoxicity, include, but are not limited to, vancomycin, cefazolin, ampicillin-sulbactam, and clindamycin. In other embodiments, antibiotics for treatment of Gram− infections, based on bactericidal activity and minimal cellular cytotoxicity, include, but are not limited to, imipenem, ceftriaxone, ceftazidime, and other carbapenem class antibiotics.

In certain embodiments, antibiotic concentrations of use to treat bacterial or drug-resistant bacterial infections of joints (e.g. acute or chronic septic arthritis, infected tendons or ligaments or associated soft tissues) as well as infections of prosthetic implants in these sites can range from about 1 mg to about 1,000 mgs. In some embodiments, the antibiotics can be administered to the same site immediately before, together with, or immediately after administration of the activated MSCs or iMSCs. In some embodiments, the activated MSCs are frozen and stored along with antibiotics and/or antimicrobial factors in a vial or bag or other container for later direct injection into the infected joint or tendon or ligament or associated soft tissues, using a syringe and needle or other delivery device.

In some embodiments, MSCs can be obtained or derived from any source known in the art, for example bone marrow, adipose tissue, cord blood (e.g. Wharton's jelly), tissue biopsies, skin biopsies, dental biopsies or induced pluripotent stem cells (iPSC). In the other embodiments, the MSCs can be derived from tissues of young (<30 yrs.) related or unrelated donors (allogeneic MSC). In some embodiments, allogenic MSCs can be derived from unrelated donors of the same species (allogeneic), or from the same individual (autologous) or from other species. In certain embodiments, compositions or combination compositions disclosed herein for administering to an infected joint, tendon, ligament or associated soft tissue can include administering a single fixed dose of about 1.0 to about 10.0×10⁷ (e.g. 2.0×10 6) activated allogeneic MSC or activated iMSCs, together with a selected bactericidal and non-cytotoxic antibiotic, having a fixed dose of about 1.0 mg to about 1000 mg (e.g. 100 mg), injected as a single combined formulation.

In other embodiments, MSCs or iMSC can be incubated in culture in the presence of one or more TLR3 ligands for about 30 minutes or up to 12 or 24 hours in order to activate the MSCs for improved anti-microbial, and immune modulatory activity for inducing bacterial clearance, reducing bacterial burden and disrupting bacterial biofilms, and overcoming antibiotic resistance. In accordance with these embodiments, activated MSCs or iMSC can be given together with bactericidal, non-cytotoxic antibiotics or to treat acute or chronically infected joints, tendons, or ligaments or associated soft tissues, or infections involving prostheses implanted in the above sites.

In certain embodiments, an infected tissue (joint, tendon, ligament or associated soft tissue) can be infected with one or more bacteria including, but not limited to, Staphylococci (e.g. S. aureus), streptococci, Haemophilus influenza, Enterobacter, Gram negative bacilli such as E. coli, Gonococcus, Pseudomonas, Klebsiella, Acinetobacter, and/or other gram+ and gram− bacteria and/or other non-cell wall containing bacteria such as mycoplasmas. In some embodiments, the infection can also include highly drug resistant strains of bacteria, including, but not limited to, MRSA strains of Staphylococcus aureus. In other embodiments, the infection can be due to other types of bacteria, including mycobacteria and mycoplasmas. In other embodiments, an infection can include a fungal infection and the combination can further include at least one anti-fungal agent. In certain embodiments, infections of highly drug-resistant bacteria or chronic infection can be infected by more than one (e.g.., 2, 3, 4, 5, or more) strain or species of bacteria. In other embodiments, a microbial infection can include fungal pathogens and/or bacteria.

In other embodiments, compositions and methods disclosed herein can be combined with other compositions and methods known in the art to treat chronic or acute infections and/or drug-resistant bacteria. In some embodiments, compositions and methods disclosed herein can be used to treat subject having infected joints or prosthetics with an underlying condition such as rheumatoid arthritis, immunocompromised, undergoing chemotherapy for cancer, type I or type II diabetes or other underlying condition.

In certain embodiments, a subject is a human such as an adult, child or infant or other mammal such as a companion animal (e.g. dog or cat or pig or goat), a horse, a livestock animal, or other mammal or animal. In certain embodiments, animals that can benefit from compositions and methods disclosed herein include, but are not limited to, horses and includes performance horses; for example, race horses, rodeo horses, hunter/jumpers, breeders, other show horses or other performance horses.

In other embodiments, compositions and methods disclosed herein can be combined with other adjunctive treatments including surgical lavage or surgical removal of any implanted prostheses and materials.

Other features and advantages of the application will become apparent from the following detailed description in conjunction with the examples.

### BRIEF DESCRIPTION OF THE DRAWINGS

FIGS. 1A-1B represent graphs analyzing synovial fluid parameters obtained from a joint in treated and untreated animals using activated MSCs over a period of days. 1A illustrates total nucleated cell count and 1B represents total protein reflective of effects of the treatment on infection of some embodiments disclosed herein.

FIG. 2 illustrates exemplary graphs reflecting quantitative synovial fluid cultures reflecting bacterial colony count in treated and untreated joints with activated MSCs of some embodiments disclosed herein.

FIGS. 3A-3B represent exemplary graphs illustrating circumference of the tarsocrural joint (effusion) in treated versus control untreated limbs determined from point of the calcaneus to distal aspect of the ridges of the trochlear tali of the animals in the animal model tested herein in some embodiments disclosed herein.

FIGS. 4A-4F represents exemplary graphs illustrating assessment of five parameters scored 0-3 (maximum total score 15: 3×5), including 1) physical examination parameters (temperature, pulse rate, respiratory rate), 2) periarticular swelling, 3) periarticular heat determined by thermography, 4) lameness, and 5) distal limb edema illustrating parameters of pain and inflammation scoring in treated versus untreated control limbs of horses. 4A represents total pain score; 4B represents Lameness; 4C Periarticular swelling; 4D TPR indications; 4E periarticular heat represented by pain score; and 4F distal limb edema of some embodiments disclosed herein.

FIG. 5 represents a graph illustrating body temperature of treated versus control untreated experimental animals over time of some embodiments disclosed herein.

FIG. 6 illustrates tubes of representative samples of synovial fluid as an indication of inflammation and infection of untreated versus treated animals, as illustrated by extent of cloudiness of some embodiments disclosed herein.

FIG. 7 is a representative photograph of plates of synovial fluid obtained from treated and untreated animals. The plates indicate bacterial counts remaining in untreated and treated animal synovial fluid where the untreated animal samples are illustrated in the upper plate and the treated animal samples are illustrated in the lower plate of some embodiments disclosed herein.

FIG. 8 is a representative photograph of plates of samples obtained from activated MSC treated and untreated joints plated out for bacterial contamination analysis with treated joints illustrated on the left and control treated joints illustrated on the left of some embodiments disclosed herein.

### DEFINITIONS

As used herein, the terms “comprises,” “comprising,” “containing,” “having,” and the like can have the meaning ascribed to them in U.S. patent law and can mean “includes,” “including,” and the like; the terms “consisting essentially of or “consists essentially” likewise have the meaning ascribed in U.S. patent law and these terms are open-ended, allowing for the presence of more than that which is recited so long as basic or novel characteristics of that which is recited are not changed by the presence of more than that which is recited, but excludes prior art embodiments.

Unless specifically stated or obvious from context, as used herein, the terms “a,” “an,” and “the” are understood to be singular or plural.

As used herein, the term “about,” can mean relative to the recited value, e.g., amount, dose, temperature, time, percentage, etc., for example, plus or minus 10 percent, ±10%, ±9%, ±8%, ±7%, ±6%, ±5%, ±4%, ±3%, ±2%, or ±1%.

As used herein, the terms “treat”, “treating”, “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

As used herein, “preventing” as used herein can mean preventing in whole or in part, or ameliorating or controlling, or reducing or halting the production or occurrence of the thing or event, for example, the disease, disorder or condition, to be prevented.

As used herein the phrases “therapeutically effective amount” and “effective amount” and the like, can indicate an amount necessary to administer to a subject, or to a cell, tissue, or organ of a patient, to achieve a therapeutic effect, such as an ameliorating or alternatively a curative effect. The effective amount is sufficient to elicit the biological or medically positive response in a cell, tissue, reversal of infection, system, animal, or human that is being sought by a researcher, veterinarian, medical doctor, health professional, or clinician. Determination of the appropriate effective amount or therapeutically effective amount is within the routine level of skill in the art.

As used herein, the terms “administering”, “administer”, “administration” and the like, can refer to any mode of transferring, delivering, introducing, or transporting a therapeutic agent compositions disclosed herein to a subject in need of such a composition. Such modes include, but are not limited to, oral, topical, intra-articular or other direct application.

As used herein, the term “Toll-like receptors (TLRs)” can refer to a class of proteins that play a key role in the innate immune system. These proteins are typically expressed in sentinel cells of the immune system such as macrophages and dendritic cells, as well as other cells including, but not limited to, mesenchymal stromal cells. They recognize structurally conserved molecules derived from microbes. Examples of TLRs include TLR 3 and TLR 4, and the like of use in activating MSCs or iMSCs using various TLR ligands disclosed herein and known in the art.

### DETAILED DESCRIPTION

In the following sections, various exemplary compositions and methods are described in order to detail various embodiments of the invention. It will be obvious to one skilled in the art that practicing the various embodiments does not require the employment of all or even some of the specific details outlined herein, but rather that concentrations, times and other specific details may be modified through routine experimentation. In some cases, well known methods, or components have not been included in the description.

Acute and chronic infections continue to be a major source of morbidity and mortality, driven in part by the increasing prevalence of drug-resistant bacteria and side effects due to these infections. In certain embodiments, compositions and methods disclosed herein concern providing solutions for issues of side effects of these infections such as deformity, temporary and permanent debility, and in many cases morbidity. In certain cases, these side effects due to drug-resistant and multi-drug resistant infections can lead to for example, septic arthritis and osteomyelitis in joints leading to the need for prosthetic implants of affected individual subjects. In some cases, prosthetic implants can become infected at adjoining tissues creating difficult to access sites of infection.

Embodiments of the present invention generally relate to compositions and methods for treating acute and/or chronic infections of joints, tendons, or ligaments, or associated soft tissues. In other embodiments, compositions and methods disclosed herein concern treating conditions related to prosthetic implant infections or infections associated with implantation of catheters or other devices. In other embodiments, compositions of use herein generally relate to compositions including activated mesenchymal stromal cells (MSCs) or activated iMSCs in a combination formulation with bactericidal, non-cytotoxic antibiotics for the treatment of infected joints, tendons, ligaments, infected prosthetics or associated soft tissues as a combination formulation.

In certain embodiments, compositions disclosed herein can be directly introduced into the site of infection of joints, tendons, ligaments, infected prosthetics or associated soft tissues to treat the subject with respect to the infection. In accordance with these embodiments, infected regions can include, but are not limited to, infections of joints, tendons, ligaments, or placement of prosthetic implants in knees, hips, and shoulders for example. In certain embodiments, the condition to be treated in a subject can include septic arthritis. In other embodiments, localized treatments of acute or chronically infected joints as well as other regions having reduced vascularization, reduced accessibility for treatment while harboring a chronic infection, or drug-resistant or multi-drug resistant infection are contemplated for treatments disclosed herein. In certain embodiments, the condition to be treated by localized treatment can include providing a mixture of activated MSCs and at least one bactericidal, non-cytotoxic antibiotic directly to a catheter for example, an indwelling bladder or bowel catheter or PIC line or other affected area.

In certain embodiments, an infection contemplated herein can be characterized by the type of infection such as the type of bacterial infection (or fungal infection). In accordance with these embodiments, an infection contemplated herein can be characterized by the type of infection such as the type of bacterial infection (or fungal infection) before, during or after a composition is administered to the subject in order to assess the type(s) of antibiotic to be provided such as Gram−, Gram+ or a mix of Gram− and Gram+ antibiotics; or other types of bacteria including mycoplasmas and mycobacteria.

In some embodiments, Toll-like Receptor-3 (TLR3) ligands can be used to activate MSCs or iMSCs of use in compositions for directly administering to a joint or tendon or ligament or prosthetic or catheter or associated soft tissues. In accordance with these embodiments, TLR3 ligands can include, but are not limited to, one or more of polyadenylic-polyuridylic acid (poly(A:U), polyinosine-polycytidylic acid (pIC), and UV-inactivated viral particles (e.g., poxvirus particles or other DNA viral particles) or any other agent capable of activating and/or binding to the TLR3 receptor to induce activation of the cells. In some embodiments, TLR3 ligands of use disclosed herein can include at least pIC. In some embodiments, pIC activation of MSCs or iPSCs can include exposure to pIC in culture for about minutes to less than 12 hours.

In certain embodiments, using TLR3 ligands for MSC or iMSC activation can include, but are not limited to, one or more of polyadenylic-polyuridylic acid (poly(A:U), polyinosine-polycytidylic acid (pIC), and UV-inactivated viral particles (e.g., poxvirus particles or other DNA viral particles) or any other agent capable of activating and/or binding to the TLR3 receptor to induce activation of the cells. In certain embodiments, a TLR3 ligand includes, but is not limited to, pIC. In other embodiments, the concentration of the TLR3 ligand can be about 1.0 μg/ml to about 100.0 μg/ml, or about 1.0 μg/ml to about 80.0 μg/ml , or about 5.0 μg/ml to about 50.0 μg/ml , or about 5.0 μg/ml to about 30 μg/ml, or about 5.0 μg/ml to about 20 μg/ml, or about 10.0 μg/ml in media for activating cells of use herein. In some embodiments, MSC or iMSC activation with pIC can include incubation for 30 minutes to about 4 hours, or about 1 hour to about 3 hours, or about 2 hours, using high molecular weight (HMW) pIC, at a concentration of about 1.0 ug/ml to about 30 ug/ml, or about 5.0 ug/ml to about 20 ug/ml, or about 10 ug/ml with cells in culture for about 2 hours. In certain embodiments, the activating TLR3 ligand can be removed or washed off of the activated MSCs or iMSCs prior to use of the activated MSCs or iMSCs disclosed herein. In other embodiments, activated cells can be washed off, collected, counted and frozen in commercial freezing medium (e.g., Cryostore plus) or other suitable medium for later use and storage. In some embodiments, activated MSCs or iMSCs can be delivered to the subject at about 1×10⁵ to about 1×10⁷ or about to 2×10⁶ cells per joint or tissue injected (e.g. not body weight) in combination with antibiotics contemplated of use herein. In some embodiments, MSCs can be obtained or derived from any source known in the art, for example bone marrow, adipose tissue, cord blood, tissue biopsies, skin biopsies, dental biopsies or induced pluripotent stem cells (iPSC), unrelated donor mesenchymal stromal cells, or other mesenchymal stromal cells. In certain embodiments, MSCs can be autologous, allogeneic or xenogeneic. In some embodiments, allogenic MSCs can be mammalian MSCs from a human, other mammals, such as companion animals, horses, or livestock or other appropriate source depending on the subject being treated and/or the condition of the subject (e.g. immunocompromised, age, other condition). In other embodiments, cells can be obtained or derived from adipose-tissue and be allogeneic from a relatively young (e.g. <30 years for a human subject) donor.

In some embodiments, direct injection of activated MSCs or iMSCs mixed with bactericidal, non-cytotoxic antibiotics or other anti-bacterial agent, either as fresh or frozen cells, mixed with antibiotics during expansion or differentiation, where the cells are infused with the anti-bacterial agents, before or after freezing or just prior to injection. In other embodiments, a treatment composition disclosed herein can be introduced directly into the affected tissue, either by intra-articular injection (joints) or direct injection into infected tendons, ligaments, or associated soft tissues. In other embodiments, the composition can be injected via a catheter implant or comparable device administered once daily over a shortened or prolonged period (e.g. minutes to several hours), a couple of times a day, every other day, three times per week, bi-weekly, weekly, every other week, every third week, monthly, every other month or other pre-determined regimen for treating the chronic or acute infection. In certain embodiments, a single injection or single catheter administration of a bolus of activated MSC or activated iMSCs plus bactericidal, non-cytotoxic antibiotic composition is anticipated for complete treatment. In other embodiments, other treatment regimens can include multiple treatments over an extended time-period to completely resolve an infection.

In some embodiments, intra-articular (IA) administration of activated MSCs or activated iPSCs in combination with one or more anti-microbial therapies, bactericidal, non-cytotoxic antibiotics applied to joints or prosthetic implants in a subject are contemplated herein. In accordance with these embodiments, IA administration of compositions disclosed herein can be to a subject in need thereof by infusion over an extended period, by predetermined timing through a catheter implant or comparable device administered once daily, every other day, three times per week, bi-weekly, weekly, every other week, every third week, monthly, every other month or other pre-determined regimen. In certain embodiments, a single IA administration of activated MSCs or activated iPSCs in combination composition with antibiotics or with at least one accompanying antibiotic treatment is sufficient to treat a targeted region. In other embodiments, two or more IA administrations of compositions disclosed herein may be needed in order to treat an affected area. In other embodiments, antibiotics include bactericidal and non-cytotoxic antibiotics administered directly with activated MSCs or iMSCs in a single fixed dose directly into an affected site. In accordance with these embodiments, antibiotics and/or other antimicrobial factors (e.g. antimicrobial peptides) can be injected directly together with the activated MSC or iMSC for maximal benefit. In certain embodiments, combination compositions of activated MSCs or iMSCs and a bactericidal, reduced or non-cytotoxic antibiotic can be administered directly in a single syringe or bolus as a combination composition to an affected site (e.g. prosthetic joint infection (PJI)).

In some embodiments, one or more antimicrobial peptide can be included in compositions disclosed herein. In accordance with these embodiments, one or more antimicrobial peptide can include, but are not limited to, LL-37, beta-defensin, CXCL10, surfactant A or the like. These compositions are contemplated of use as combination compositions for the treatment of an infection by direct administration to an infected site, region or joint or implant device. In some embodiments, an antimicrobial peptide can be combined with activated MSCs or activated iMSCs and antibiotics disclosed herein to treat an infection in a joint, tendon, ligament or infected implant.

In some embodiments, antibiotics for treatment of Gram+ infections, based on bactericidal activity and minimal cellular cytotoxicity, include, but are not limited to, vancomycin, cefazolin, ampicillin-sulbactam, and clindamycin. In certain embodiments, vancomycin, or other Gram+ targeted antibacterial agent can be combined directly with activated MSCs or activated iMSCs. In other embodiments, antibiotics for treatment of Gram− infections, based on bactericidal activity and minimal cellular cytotoxicity, include, but are not limited to imipenem, ceftriaxone, ceftazidime, and other carbapenem class antibiotics. In some embodiments, the one or more antibiotic include, but are not limited to, one or more of vancomycin, or other gram positive bacteria-directed antibiotic or imipenem, or other gram negative bacteria-directed antibiotic or antibiotic against both gram positive and gram positive bacteria. In other embodiments, antibiotics of use in compositions disclosed herein can include vancomycin, ampicillin sulbactam, or amikacin. In some embodiments, antibiotics of use in compositions disclosed herein can include vancomycin to treat gram positive (Gram+) infections and/or ceftazidime or imipenem to treat gram negative (Gram−) infections in combination compositions with activated MSCs and/or activated iMSCs for direct administration to drug resistant or multi-drug resistant bacterial infections in a targeted region such as a joint, tendon or implant region, bone abscess, or soft tissue infected region.

In some embodiments, implants or a subject receiving an implant can be pretreated with combination compositions disclosed herein to reduce onset of an infection in a recipient. In other embodiments, catheters or a subject receiving a catheter can be pretreated with combination compositions disclosed herein to reduce onset of an infection in a recipient at the intended site of implantation or surrounding tissues.

In some embodiments, antimicrobial agents such as antibiotics of use in combination formulations disclosed herein exclude antibiotics having cytotoxic effects towards mammalian cells, including MSC, iMSC, and connective tissue cells of joints and soft tissues. In accordance with these embodiments, excluded antimicrobial agents include, but are not limited to, aminoglycosides, fluoroquinolones, tetracyclines, and neomycin. In other embodiments, antimicrobial agents that are bacteriostatic rather than bactericidal can be excluded, such as antibiotics of use in combination formulations disclosed herein exclude tetracyclines, macrolides, sulfonamides, lincosamides, trimethoprim, chloramphenicol, and rifampin. In yet other embodiments, antimicrobial agents such as antibiotics having negative effects on cells such as activated MSCs disclosed herein of use in combination formulations exclude aminoglycosides, fluoroquinolones, tetracyclines, neomycin, macrolides, sulfonamides, lincosamides, trimethoprim, chloramphenicol, and rifampin.

In certain embodiments, vancomycin can be used together with activated MSCs or activated iMSCs to treat gram positive (Gram+) infections with reduced cytotoxicity. In some embodiments, the antibiotic can be mixed and frozen with activated MSCs or activated iMSCs for storage, transport, and later use. In other embodiments, activated MSCs or activated iMSCs plus imipenem can be used to treat gram negative (Gram−) infections with reduced cytotoxicity. In some embodiments, antibiotics having reduced cytotoxicity can be co-administered directly with the activated MSCs or activated iMSCs, immediately at the time of treatment. In other embodiments, the cells and antibiotics can be initially administered as a combination composition and then follow-up treatments can be administered separately, and in certain methods, within minutes apart such as less than 10 or less than 5 minutes apart from one another.

In certain embodiments, antibiotic concentrations of use to treat bacterial or drug-resistant bacterial infections of joints, tendons, ligaments, implants, or catheters or associated soft tissues can range from about 1 mg to about 1,000 mgs. In accordance with these embodiments, antibiotic concentrations can range from about 20 mg to about 500 mg, or about 20 mgs to about 250 mgs, or about 20 to about 200 mgs, or about 50 to about 150 mgs or about 100 mgs per dose depending on the subject to be treated, the severity of infection, the dosing regimen to be followed (e.g. continuous, daily, weekly etc.) and other factors. In certain embodiments, the antibiotics are administered directly together with the activated MSC or iMSCs into the affected joint, tendon or tissue site. In certain embodiments, the antibiotic concentration is from about 20 mg to 200 mgs together with activated MSCs or activated iMSCs.

In certain embodiments, antibiotic concentrations of use to treat bacterial, mixed bacterial or drug-resistant bacterial infections of joints (e.g. septic arthritis, acute or chronic septic arthritis) as well as infections of implants can range from about 1 mg to about 1,000 mgs. In accordance with these embodiment, antibiotic concentrations can range from about 20 mg to about 500 mg, or about 20 mgs to about 250 mgs, or about 20 to about 200 mgs, or about 50 to about 150 mgs or about 100 mgs per dose depending on the subject to be treated, the severity of infection, the dosing regimen to be followed (e.g. continuous, daily, weekly etc.) and other factors. In some embodiments, the antibiotics can be administered before, during or after administration of the activated MSCs or activated iPSCs. In other embodiments, antibiotics can be combined with the activated MSCs or activated iPSCs in a vial, tube, syringe or other applicable device for transport, storage or intra-articular (IA) injection or direct injection and administration directly to the affected area such as the joint or implant region.

In yet other embodiments, activated MSCs in concentrations disclosed herein can be administered by direct injection to the affected area while the subject is being administered an uninterrupted dose of antibiotics by infusion or direct administration in a single composition. In yet other embodiments, antibiotics and activated cells disclosed herein can be provided alone or together in a timed-delivery device or medium such as microparticles, a porous gelatinous material or other suitable delivery method for timed or continuous delivery of the agents to the region while maintaining localized treatment of the compositions. In accordance with these embodiments, time-delivery type devices housing compositions disclosed herein can be administered or placed directly in the affected region of the subject. In certain embodiments, combinations of treatments disclosed herein work in concert to enhance antibiotic activity, disrupt bacterial biofilms, induce recruitment and activation of immune effector cells (e.g., monocytes, neutrophils) for increased antimicrobial activity,

In some embodiments, MSCs can be obtained or derived from any source known in the art, for example bone marrow, adipose tissue, cord blood, Wharton's jelly, tissue biopsies, skin biopsies, dental biopsies or induced pluripotent stem cells (iPSC) (iMSC), unrelated donor mesenchymal stromal cells, or other mesenchymal stromal cells from other species. In some embodiments, allogenic MSCs can be derived from unrelated donors of the same species, appropriate source depending on the subject being treated and/or the condition of the subject (e.g. immunocompromised, age, other condition). In some embodiments, allogenic MSCs can be mammalian MSCs from a human, other mammal such as a pet or livestock or other appropriate source depending on the subject being treated and/or the condition of the subject (e.g. immunocompromised, age, other condition).

In certain embodiments, compositions or combination compositions disclosed herein for administering to an affected joint and/or prosthetic can include administering in a single administration activated MSCs or activated iMSCs and antibiotics having reduce cytotoxicity. In accordance with these embodiments, a single administration of activated MSCs or activated iMSCs can include about 1.0×10⁶ to about 10×10⁷ or about 1.0×10⁶ to about 70×10⁶ or about 5.0×10⁶ to about 50×10⁶ or about 10×10⁶ to about 30×10⁶ cells or about 20.0×10⁶ cells per treatment in combination with antibiotics disclosed herein.

In some embodiments, vancomycin and/or imipenem can be introduced directly along with activated MSCs or activated iPSCs to the subject at a dose of about 50 mg to about 200 mg, or about 75 to about 150 mg, or about 100 mg per injection, together with activated MSCs or activated iMSCs cells from about 1.0×10⁶ to about 10×10⁷ in about 5-10 ml or other appropriate volume depending on mode of delivery and area to be treated in a subject. In some embodiments, administration can be given by direct injection. In other embodiments, vancomycin and/or imipenem and/or ceftazidime can be introduced to the site of infection at a dose of about 50 to about 200 mg or about 100 mg while activated MSCs or iMSC can be administered to the same subject at about a dose of 5 to 50×10⁶ cells per treatment in a single application or over a single treatment regimen. It is understood that the treatments can be adjusted to optimize treatment depending on the subject to be treated and the region affected in the subject to be treated. In certain embodiments, doses disclosed herein are directed to humans and livestock such as horses or cattle or similar-sized subjects. For smaller species, fixed dosing (not concentrations) of the combinations can be adjusted by reduced volume or other reduced dosing in order to treat a smaller species effectively and without adverse effects. In some embodiments, combination composition treatments are provided to a subject in need twice weekly or once a week or as needed to reduce or eliminate infection in the subject.

In other embodiments, to activate MSCs or iMSC, the cells can be incubated in culture in the presence of one or more TLR3 ligands for about 30 minutes, or about an hour, or about 1.5 hours, or about 2 hours or about 3 hours, or about 4 hours, or up to 12 or 24 hours in order to activate the MSCs or iPSCs. In accordance with these embodiments, activated MSCs or iMSCs have improved anti-microbial, immunostimulatory or other relevant effects such as inducing bacterial clearance, reducing bacterial burden, or inducing bacterial clearance and reducing bacterial burden. In accordance with these embodiments, activated MSCs or iMSC can be used alone, together or alternating activated MSCs and iMSC to treat acute or chronically infected joints or prosthetics or other implanted device for improved outcomes alone or in combination with anti-microbial agents. In some embodiments, the population of MSCs can be activated by in vitro and/or ex vivo incubation with the one or more TLR3 ligands.

In certain embodiments, a bacterial infection or drug-resistant bacterial infection of a joint, tendon and/or a prosthetic implant can include, but is not limited to, Staphylococci (e.g. S. aureus), streptococci, Haemophilus influenza, Enterobacter, Gram negative bacilli such as E. coli, Gonococcus, Pseudomonas, Klebsiella, Acinetobacter, and/or other gram+ and gram− bacteria, acid-fast positive and/or other non-cell wall containing bacteria. In some embodiments, the infection can also include highly, drug resistant strains of bacteria including MRSA strains of Staphylococcus aureus. In other embodiments, the infection can be due to other types of bacteria, including mycobacteria and mycoplasmas. In some embodiments, an infection can include a fungal infection and the combination can be used alone or can further include at least one anti-fungal agent. In certain embodiments, infections of highly drug-resistant bacteria or chronic infection can be infected by more than one (e.g.., 2, 3, 4, 5, or more) strain or species of bacteria. In other embodiments, the infection can include multiple species of fungal pathogens and/or bacteria. In certain embodiments, infections of highly drug-resistant bacteria or chronic infection can be infected by more than one (e.g.., 2, 3, 4, 5, or more) strain or species of bacteria.

In some embodiments, compositions and methods disclosed herein can be used to treat subject having infected joints or prosthetics with an underlying condition such as rheumatoid arthritis, being immunocompromised, undergoing chemotherapy for cancer or other health condition, type I or type II diabetes or other underlying condition. In other embodiments, a subject contemplated herein can have a condition or an infection post a surgical event where a localized infection of a joint or implant region occurs.

In certain embodiments, a subject is a human such as an adult, child or infant or other mammal such as a pet, livestock, or other animal. In certain embodiments, large animals that can benefit from compositions and methods disclosed herein include, but are not limited to, horses and includes performance animals, for example, race or endurance horses, hunter/jumpers, breeders or other performance or high performance horses or competitive horses. In other embodiments, the animal is a wild animal or animal in captivity. In certain embodiments, the treated individual is a human being, either an adult, adolescent or a child or infant. In other embodiments, the treated individual is a mammal such as any pet, a dog, a cat, a goat, a rabbit, other livestock, zoo animals or other companion, captive or wild animal.

In some embodiments, Mesenchymal Stromal Cells (MSCs) and iMSCs possess toll-like receptors (TLRs) and are involved in the inflammatory response to infection. Pre-activation of MSCs with TLR ligands trigger release of chemokines and factors that increase neutrophil survival, induce immunostimulatory effects and release factors to reduce bioburden. Enhanced antimicrobial activity of TLR3 ligands using for example, polyI:C activated vs. resting MSCs or iPSCs can improve infection control with and without antimicrobial agents. In accordance with embodiments disclosed herein, activated MSCs have superior activities compared to un-activated MSCs of use to treat an infection in joints, tendons and infected implants or catheters.

In some embodiments, direct administration of TLR3 activated MSCs (e.g. adipose-derived MSCs) or activated iMSCs to a chronic or acute infected area disclosed herein can improve clinical outcome. In other embodiments, direct administration of TLR3 activated MSCs (e.g. adipose-derived MSCs) or activated iMSCs to a chronic or acute infected area combined with antibiotics can improve clinical outcomes and reduce bacterial burden and joint inflammation of septic arthritis in a subject compared to conventional antimicrobial therapy alone. In accordance with these embodiments, certain parameters can be evaluated to test treatment efficacy and to evaluate the need for additional treatments; for example, synovial fluid or other samples such as soft tissue or joint sample can be obtained from a subject. In accordance with these embodiments, fluid or other samples obtained from a subject can be analyzed for one or more of bacterial analysis. markers of inflammation, clinical pain and function scoring or similar test in order to assess treatment success and need for additional treatments or alteration of a treatment regimen, for example.

In other embodiments, bacterial infections characterized by the development of bacterial biofilms, for example on the surface of implants such as catheters or orthopedic devices or on tissues such as cartilage or synovial linings and tendon sheaths and ligaments can be treated by compositions and methods disclosed herein. It is noted that these infections can be particularly difficult to manage with antibiotic therapy alone, often requiring weeks to months of continuous therapy if even successful which with chronic infection is often not successful. In certain embodiments, despite aggressive antibiotic therapy, in many cases biofilm-infected devices or implants must be removed to fully resolve these chronic infections or can lead to drastic consequences including death. In accordance with these embodiments, compositions and methods disclosed herein can be used to treat these infections where TLR3 activated MSCs or activated iMSCs can be combined with bactericidal and non-cytotoxic antibiotics and directly administrated to the affected regions, including joints, tendons, ligament, or associated tissues. In certain embodiments, activated MSCs migrate to the area to combine with bactericidal and non-cytotoxic antibiotics to treat acute and chronic infection including multi-drug resistant bacterial infections.

In some embodiments, activated MSCs, activated iMSCs alone or in combinations with bactericidal and non-cytotoxic antibiotics can be used for the treatment of chronic, traumatic, and/or post-operative implant infections that often involve multi-drug resistant bacteria that form biofilms, making treatment particularly problematic and often inaccessible. In certain embodiments, activated MSCs and antibiotic combination compositions thereof can be useful for the treatment of highly antibiotic drug resistant infections and/or infections caused by multiple strains of the same or different antibiotics.

Multiple complementary mechanisms of action likely account for the ability of activated MSCs or activated iMSCs to dramatically reduce drug-resistant bacterial infections of joints or implants in combination with antibiotics. In accordance with these embodiments, the net effect of MSC administration on bacterial burden reflects the sum of both direct and indirect mechanisms of action. One direct mechanism of anti-bacterial action demonstrated was secretion of antimicrobial peptides such as cathelicidins by activated MSCs which were localized to the region of infection. In other embodiments, one indirect mechanism was bacterial elimination by activated MSCs by interactions of activated MSC with the host innate immune response and immune cell recruitment and activation as well as proinflammatory effects.

### Mesenchymal Stromal Cells

MSCs are multipotent stem cells that are capable of differentiating into osteoblasts, chondrocytes, myocytes, and adipocytes. Under tissue culture conditions, MSCs exhibit plastic adherent properties and are most often classified as CD73+, CD90⁺, CD105⁺, CD11b⁺, CD14⁻, CD19⁻, CD34⁻, CD45⁻, CD79a⁻, and MHC class II negative cells, though it should be noted that MSC isolated from different tissue sources will display different surface phenotypes. The antimicrobial properties of MSC can vary from one tissue source to another and are therefore considered somewhat unpredictable in terms of their effectiveness for combatting bacterial infection without activation. Activation by TLR and other innate immune pathways can trigger the upregulation of secretion of these antimicrobial factors and dramatically improve the anti-microbial properties of MSCs.

MSCs are a subpopulation of cells of neural crest origin that exist in most tissues in the body in low numbers. MSCs from a variety of sources have been shown to have antimicrobial activity in sepsis, wound healing, and infections in animal models but not been demonstrated until the instant case to be useful in treating joints, tendons or implant infections contemplated herein. The presence of Toll-like receptors (TLRs) on and within MSCs and the impact of TLR ligands on MSC activity has also been studied. The results of these studies lead to a wealth of often contradictory information regarding the effects of MSC on the properties that ultimately result. For example, researchers have shown that activation of MSCs with a ligand of TLR3 increases the immunosuppressive function of MSCs, an effect that would normally be contraindicated for treatment of infected tissues such as joints and tendons. However, antibacterial properties are enhanced by activation of MSCs through TLR ligands.

Most experimental and clinical studies with MSC use cells derived from bone marrow (BM-MSC) or from adipose tissues (Ad-MSC). However, the use of BM-MSC and Ad-MSC does potentially involve drawbacks because, when autologous MSC are used, the age of the donor greatly affects the immunological and antimicrobial properties of the MSC, as cells from older patients or animals typically are less functional. As disclosed herein one source of MSCs can be from an allogeneic (unrelated) donor that is 30 years old or less for treatment of human patients. Other MSC sources are contemplated herein.

In other embodiments, one solution to this issue is the use of MSC derived from induced pluripotent stem cells (iPSC). iPSC can be generated from a number of different adult tissues and differentiated into MSC, which are referred to herein as “iMSC.” Several of the advantages associated with the use of iMSC include the fact they can be propagated indefinitely, that they regain the functionality of young MSC even if derived from older individuals, and that they can serve as a single source of cells for treatment of multiple subjects with maintained efficacy and reduced costs. In addition, improved antimicrobial activity of iMSC subpopulations can lead to select populations of use to treat subjects having bacterial infections of joints, tendons, ligaments, infected prosthetics or associated soft tissues.

### Activation of MSCs

While MSC are able to secrete antimicrobial factors spontaneously in culture, their secretion of these factors can be markedly upregulated by activation with TLR ligands, for example, with TLR3 ligands. MSC activation can occur by in vitro incubation of the MSCs with an effective amount of the activating agent. For example, the MSCs can be incubated with the activating agent (e.g., TLR3 ligand)) for about 30 minutes, or about 1, 2, 3, 4, 5, 6, 7, 8, 9, 10 or up to 12 hours or more but less than 24 hours. In accordance with these embodiments, concentrations of the activating agent can be about 1.0, 2.0, 3.0, 4.0, 5.0, 6.0, 7.0, 8.0, 9.0, 10.0, 15.0, 20.0, 25.0 or more μg/ml are contemplated. In one embodiment, MSCs can be activated by in vitro incubation with about 1.0 to about 30 μg/ml pIC or other TLR3 ligand for 30 minutes to less than 24 hours; less than 12 hours; less than 10 hours or less than 6 hours or about 1 hr to less than 6 hours. In another embodiment, MSCs can be activated by in vitro incubation with about 5.0 to about 15.0 μg/ml pIC for 30 minutes to less than 6 hours or about 2 hours.

### Administration

An effective number or concentration of activated MSCs, activated iMSC or any of the compositions described herein can be administered to a subject administration by direct injection, using a needle and syringe, directly into infected joints, tendons, ligaments, or associated soft tissues. Any suitable administration protocol known in the art can be used to administer the activated MSCs and antibiotic compositions disclosed herein. In accordance with these embodiments, activated MSCs+antibiotic compositions and/or other disclosed compositions can be administered as a single dose or can be administered in multiple (e.g., 2, 3, 4, 5, 6, 7, 8, 9, 10, or more) doses.

In some embodiments, a subject can receive one or more than one (e.g., 2, 3, 4, 5, 6, 7, 9, 10, 11, 12, 13, 14, 15, 16, 17, 18, 19, 20, or more) doses of any of the compositions described herein that include activated MSCs, activated iPSCs or combinations of activated cells with an antibiotic having reduced cell-killing properties. When more than one dose is to be administered, the doses can be administered at regular or irregular intervals. In some embodiments, the doses can be administered daily, weekly, biweekly, monthly, or bimonthly or on a predetermined maintenance schedule to reduce the chance of or prevent recurrence of infections. For example, the doses can be administered two times daily, every 1, 2, 3, 4, 5, 6, or 7 days; every 1, 2, 3, 4, 5, 6, 7, 8, 9, 10, 11, or 12 weeks; and/or any combination thereof. In one embodiment, three separate doses (e.g., three treatments) can be administered over a 6-week period (e.g.., every two weeks).

In some embodiments, a subject can receive one or more than one (e.g., 2, 3, 4, 5, 6, 7, 9, 10, 11, 12, 13, 14, 15, 16, 17, 18, 19, 20, or more) doses of any of the compositions described herein that include activated MSCs, activated iPSCs or combinations of activated cells with an antibiotic. When more than one dose is to be administered, the doses can be administered at regular or irregular intervals. In some embodiments, the doses can be administered daily, weekly, biweekly, monthly, or bimonthly or on a predetermined maintenance schedule to reduce the chance of or prevent recurrence of infections. For example, the doses can be administered every 1, 2, 3, 4, 5, 6, or 7 days; every 1, 2, 3, 4, 5, 6, 7, 8, 9, 10, 11, or 12 weeks; and/or any combination thereof In one embodiment, three separate doses (e.g., three treatments) can be administered over a time period such as a 6-week period (e.g.., every two weeks). In certain embodiments, treatments can be applied to a region of an infected subject and then samples obtained some time after treatment to assess need for a subsequent treatment and scheduling. In certain embodiment, treatments can be administered over a continuous period of time or at intervals to a subject until an infection is cleared. Any method for assessing infection and types of microorganisms present in an infected region are contemplated herein.

### Compositions

Provided herein are activated MSCs or iMSCs combined with antibiotics in or along with one or more pharmaceutically acceptable carriers, diluents, excipients, or vehicles.

The terms “pharmaceutically acceptable” refer to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio.

A pharmaceutical composition of the instant disclosure is formulated to be compatible with its intended route of administration (e.g., intra-articular, intravenous, bolus, topical, transmucosal, transdermal administration). Solutions or suspensions used for administration can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

In some embodiments, pharmaceutical compositions suitable for injectable use include sterile aqueous solutions (where water soluble) or dispersions and sterile powders for the extemporaneous preparation of sterile injectable solutions or dispersions of cells. For intra-articular administration or direct injection into tissues, suitable carriers include physiological saline, bacteriostatic water, Cremophor EL™ (BASF, Parsippany, N.J.) or phosphate buffered saline (PBS). Diluents are selected to provide stable formulations for transport and storage and administration under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of microbial contamination can be achieved by various antibacterial and antifungal agents, for example, parabens, chlorobutanol, phenol, ascorbic acid, thimerosal, and the like. In some embodiments, it will be desirable to include isotonic agents, for example, sugars, polyalcohols such as mannitol, sorbitol, sucrose, trehalose, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound(s) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In some embodiments, a kit or components in a kit can include a composition for a single dose or for multiple doses. In other embodiments, a delivery device can include a catheter, a long-tipped syringe, or a device for direct tissue administration or a bulb tip or other delivery tip for intra-articular administration. In other embodiments, a syringe can be used to or is adapted for use to deliver the composition to by any delivery mode contemplated herein and at least for intra-articular administration. For example, delivery directly to the affected region. In certain embodiments, the subject is a human, or an animal such as a mammal including, but not limited to companion animals (e.g. dog and cat), horses, or livestock, or other suitable animal.

In some embodiments, activated MSCs or activated iPSCs or iMSCs either alone or in combination with one or more other therapeutic agents (e.g., one or more antibiotics), can be used in the manufacture of the medicament, for example, a medicament for treating an infected joint or implant or bone abscess or soft tissue. Any of the agents can be included in a kit contemplated herein. In addition, one or more containers are contemplated where the agents can be packaged, stored and transported separately or together within one or more containers. An intra-articular delivery device can also be included in the kit.

### Methods of Treatment

In some embodiments, use of activated MSCs or iPSCs for infected, drug-resistant infected or multi-drug resistant infected joints or implants include treatment of diabetic patients, those receiving catheters or other implanted devices or prosthetics having impaired ability to overcome an infection are contemplated. For example, the infected implants, infected catheters, and pre- and post-osteomyelitis and other chronic infections. Other indications for activated MSC for infection treatment include the treatment of infections that contain multiple species or strains of MDR bacteria, including mixed infections with Gram-negative and Gram-positive infections, and mixed infections with different Gram-positive or Gram-negative bacteria.

Methods are also provided where the compositions of activated MSC combined directly with bactericidal, non-cytotoxic antibiotics. In some embodiments, specific combinations of activated MSC or other composition described herein and bactericidal antibiotic therapy is required for efficient elimination of bacterial infection in deep tissues, and a strong synergy is observed when antibiotics and activated MSCs or activated iMSCs are combined in vivo for treatment of deep bacterial infections.

In other embodiments, activated MSCs together with bactericidal antibiotics or other compounds (e.g., antimicrobial peptides) that can disrupt bacterial biofilms. Because infections of implants associated with biofilm production are extremely difficult to eradicate with conventional antibiotic therapy, the combination treatments described herein (e.g., with activated MSCs and bactericidal antibiotics) are an advantage over the prior art. Administration of any of the compositions described herein can enhance bacterial clearance, and stimulate healing of the infected tissues. Suitable compositions and/or activated mesenchymal stem cells (whether alone or in combination with one or more bactericidal and/or bacteriostatic antibiotics) can be administered at the site of infection.

### EXAMPLES

The following examples are included to illustrate various embodiments. It should be appreciated by those of skill in the art that the techniques disclosed in the examples which follow represent techniques discovered to function well in the practice of the claimed methods, compositions, and apparatus. However, those of skill in the art should, in light of the present disclosure, appreciate that changes may be made in some embodiments which are disclosed and still obtain a like or similar result without departing from the spirit and scope of the inventions.

### Example 1

In one example, a model of septic arthritis was studied. Septic arthritis causes significant morbidity and mortality in human and animals and is increasingly complicated by the rising incidence of multidrug resistance. Certain objectives of this study were to determine if intra-articular (IA) administration of TLR activated MSCs (e.g. bone marrow derived MSCs) improve clinical outcomes and reduce bacterial burden and biomarkers of joint inflammation in an equine model of septic arthritis compared to conventional antimicrobial therapy alone. It was hypothesized that activated MSC therapy in combination with conventional antibiotic therapy could improve resolution of septic arthritis as assessed by lameness evaluation, joint circumference, imaging, and more rapid resolution of bacterial infection and inflammatory biomarkers.

In this example, four horses, each experimentally infected in one joint with MRSA strain of Staphylococcus aureus in one joint were set up for the study. Treatment started 24 h after introduction of the infective agent, two antibiotic only (control); two in TLR3 MSC plus antibiotic. All horses were treated with vancomycin (e.g. 100 mg) injected directly into the joint. The control 2 horses were just treated with vancomycin. The experimental 2 horses were (TLR MSC horses) were also treated with activated equine allogeneic MSC (e.g. 2×10⁶) injected into joint immediately after vancomycin. These treatments were repeated three times at 3-day intervals for MSC horse, only twice for control horse (euthanized on day 7). The treatment responses were evaluated by cultured synovial fluid from joints (bacti counts, cell counts) and joint circumference and swelling. The control horses were euthanized on day 7 due to pain associated with uncontrolled joint infection. The experimental horses receiving the combined treatment were euthanized on day 14 to assess joint parameters and bacterial counts. The treatment horses were nearly free of any signs of infection. The following figures represent samples collected and analyzed regarding this study. It is contemplated herein that the vancomycin and activated MSCs can be co-administered or mixed and administered as a single composition.

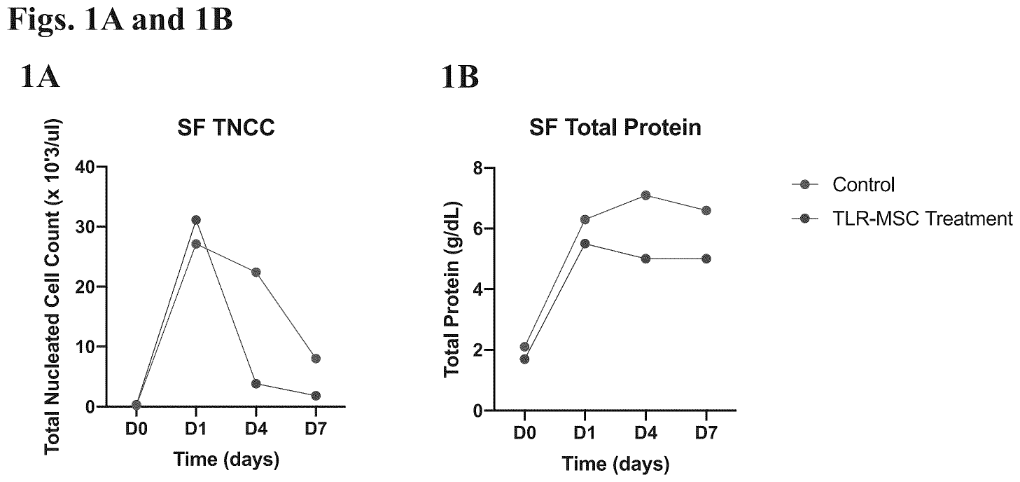

FIGS. 1A and 1B represent graphs analyzing synovial fluid parameters obtained from a joint in treated and untreated animals using activated MSCs over a period of days. 1A illustrates total nucleated cell count and 1B represents total protein reflective of effects of the treatment on infection. Synovial fluid was collected from each horse in the study, at the indicated time points, starting the day before infection (DO) and continuing until day 7. Control horses treated with vancomycin injections into the joint only, while TLR-MSC horse was treated with both activated MSC and vancomycin, both injected into the joint. The vancomycin dose was 100 mg per injection, while the dose of TLR3 activated MSC was 2×10⁶ cells per injection, consisting of activated, allogeneic MSC obtained from bone marrow of unrelated healthy horses.

Synovial fluid samples were analyzed for their total white blood cell count (TNCC) and their total protein concentration. The horse treated with TLR-MSC had substantially fewer white blood cells in the synovial fluid at later time points, as well as reduced protein concentrations, indicative of improvement in joint inflammation by reducing inflammation. Similar results were obtained in two additional study horses (data not shown).

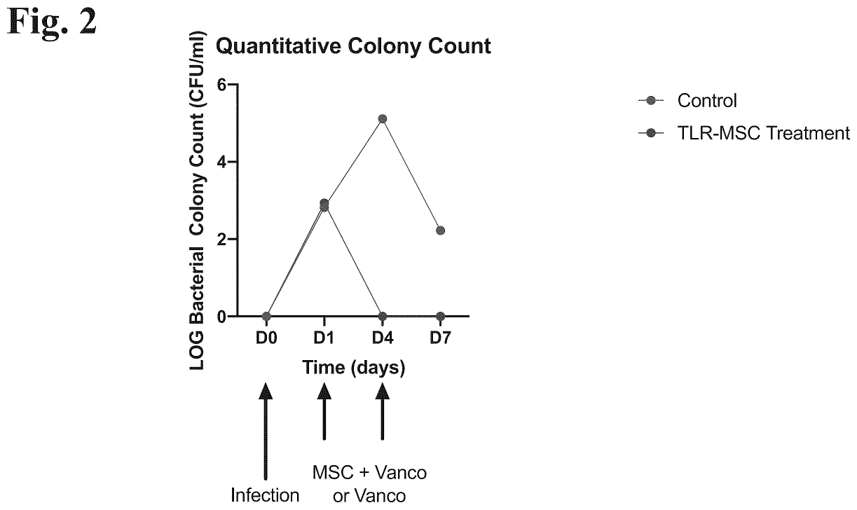

FIG. 2 illustrates graphs reflecting quantitative synovial fluid cultures reflecting bacterial colony count in treated and untreated joints with activated MSCs. Synovial fluid samples from the two horses described in FIGS. 1A and 1B were also evaluated for their concentration of live MRSA bacteria. Collected samples were processed to disaggregate the bacteria in the synovial fluid, then the sample was serially diluted in PBS and then plated on quadrant plates with LB agar. The concentration of bacteria in synovial fluid from the horses treated with TLR-MSC+vancomycin were significantly less than in the horses treated with only vancomycin through day 7, and between day 7 and day 14 no viable bacteria could be recovered from the TLR-MSC/antibiotic treated horse (not shown). Essentially identical results were obtained in two sets of study horses (data not shown but available for second set of horses).

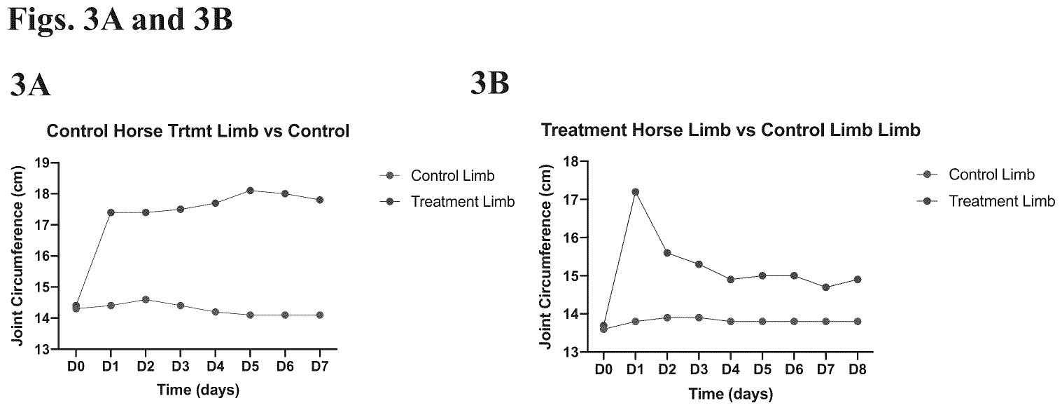

FIGS. 3A-3B represent graphs illustrating circumference of the tarsocrural joint (effusion) in treated versus control limbs determined from point of the calcaneus to distal aspect of the ridges of the trochlear tali of the animals in the animal model tested herein.

In this example, the clinical response to treatment was assessed in two horses infected with MRSA in one joint each, as noted above. Joint infection resulted in a marked increase in the circumference of the infected joint (blue, top line) compared to the uninfected joint (red, bottom line). In the left panel (horse treated only with vancomycin, top line left panel), the infected joint remained very swollen (high joint circumference, blue line, top line left panel, 3A) compared to the TLR-MSC+vancomycin treated horse (right panel, 3B, top line), in which the joint circumference began to quickly subside and return almost to normal.

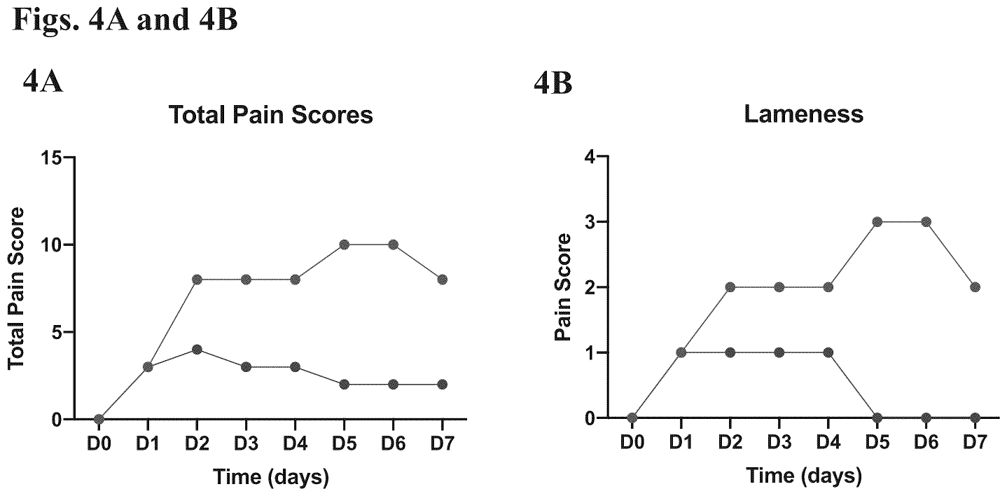

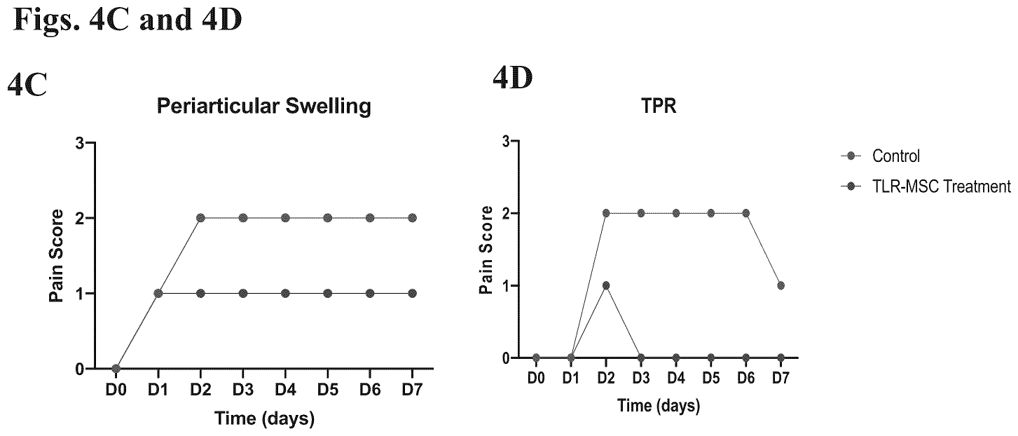

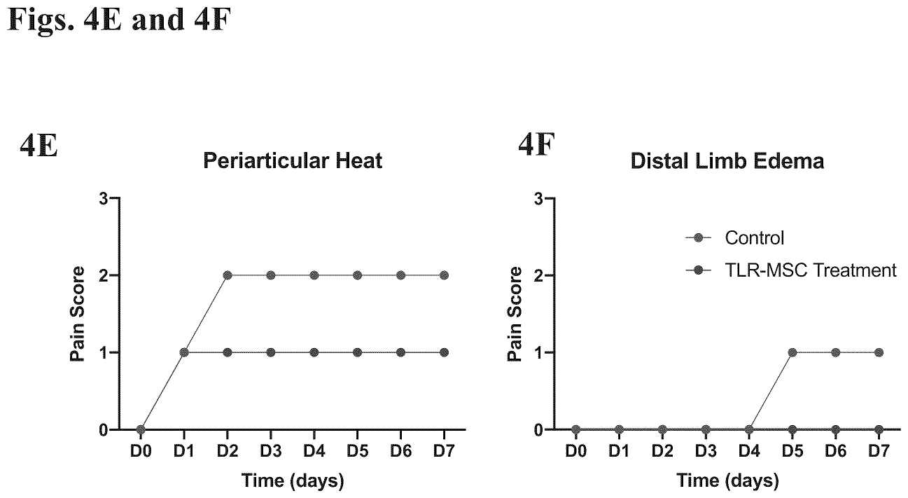

FIGS. 4A-4F represents graphs illustrating the assessment of five parameters scored 0-3 (maximum total score 15), including 1) physical examination parameters (temperature, pulse rate, respiratory rate), 2) periarticular swelling, 3) periarticular heat determined by thermography, 4) lameness, and 5) distal limb edema illustrating parameters of pain and inflammation scoring in treated versus untreated control limbs of horses. 4A represents total pain score; 4B represents Lameness; 4C Periarticular swelling; 4D TPR indications; 4E periarticular heat represented by pain score; and 4F distal limb edema.

The clinical response to joint infection in two horses was also assessed by measuring total pain scores, which consisted of lameness scores, periarticular swelling scores, temperature/pulse/respiration scores, periarticular heat scores, and distal limb edema scores. The horse treated only with vancomycin is depicted by red line (top line in all panels), while the horse treated with TLR-MSC+vancomycin is depicted by the blue line, bottom line in all panels. For all the parameters measured, the clinical scores were markedly reduced in the horse treated with TLR-MSC plus antibiotics (vancomycin) compared to the horse treated only with vancomycin. Very similar clinical scores were obtained in two additional study horses (data not shown).

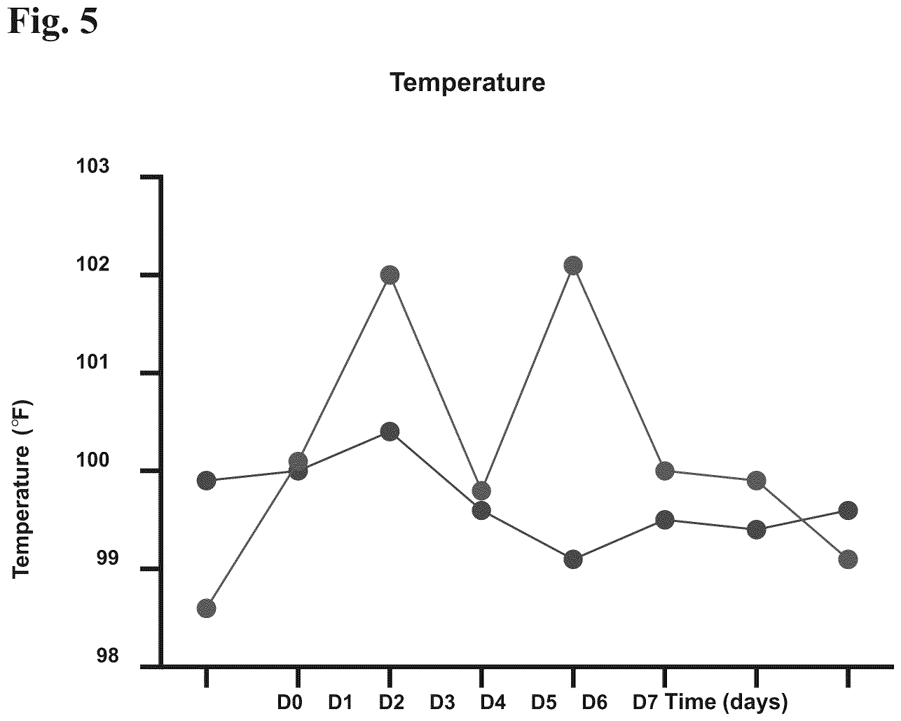

FIG. 5 represents a graph illustrating temperature of the tested animal over time in treated and untreated animal. In this example, impact of joint infection and treatment on overall body temperature (fever) were measured in two horses infected with MRSA. In the horse treated with only vancomycin (red line, top spiked line to around 102° F. in peaks), body temperature spikes were noted at two time points, while body temperature did not change in the horse treated with TLR-MSC plus antibiotic (vancomycin) (blue line, bottom steady line with near normal temperatures illustrated). Similar results were obtained in two additional study horses.

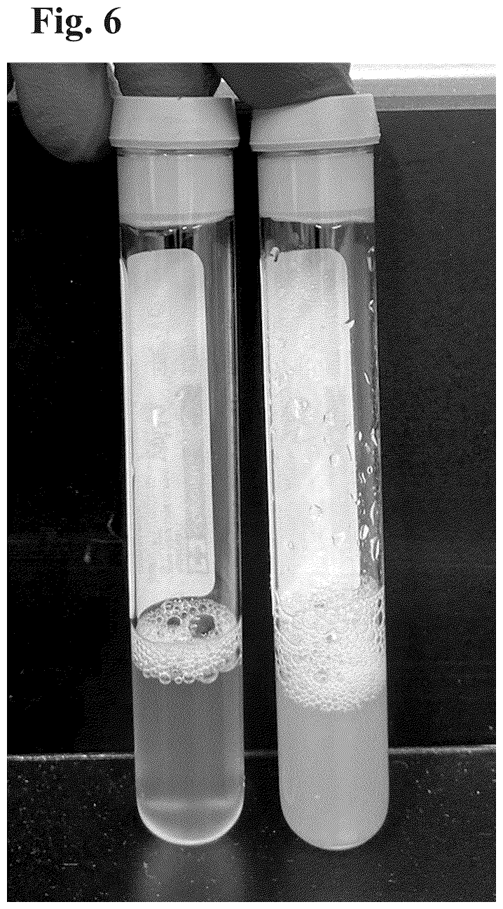

FIG. 6 illustrates tubes of representative samples of synovial fluid as an indication of inflammation, as illustrated by cloudiness of some embodiments disclosed herein. Synovial fluid was collected from two study horses, demonstrating how much cloudier fluid was found in the horse only treated with vancomycin (right tube), compared to the horse treated with activated MSC plus vancomycin (left tube). Similar results were obtained in a second study in two additional horses.

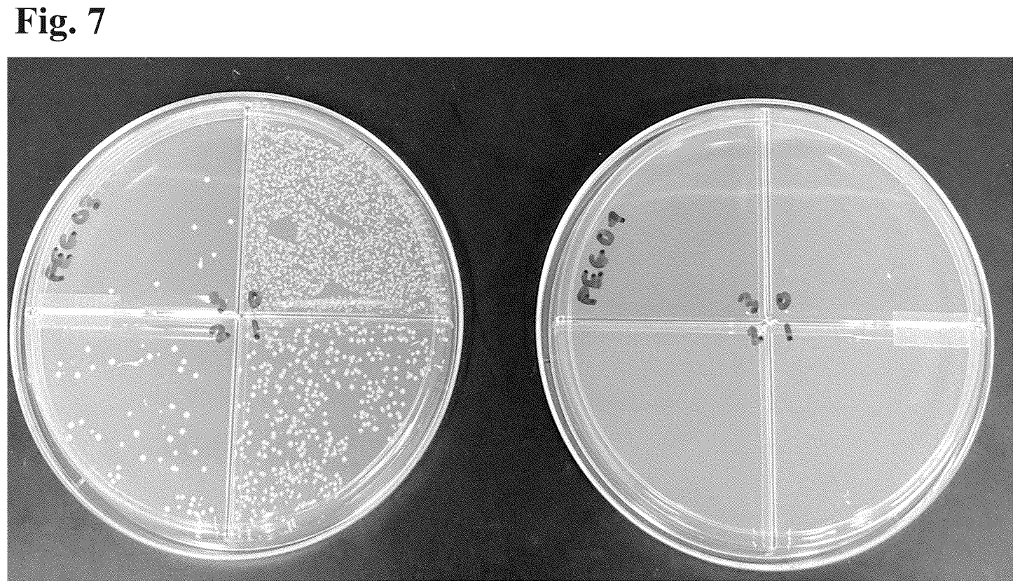

FIG. 7 is a representative photograph of plates of synovial fluid obtained from treated and untreated animals. The plates indicate bacterial counts remaining in untreated and treated animals. Synovial fluid was collected from study horses on day 4 of the study, and subjected to quantitative bacterial culture, using LB agar and quadrant plates. Bacterial colonies of MRSA appear as white dots. The density of bacteria in the synovial fluid of the horse treated with activated MSC plus vancomycin was significantly reduced compared to the density of bacteria in the horse treated only with vancomycin. Similar results were obtained in a second study in two additional horses.

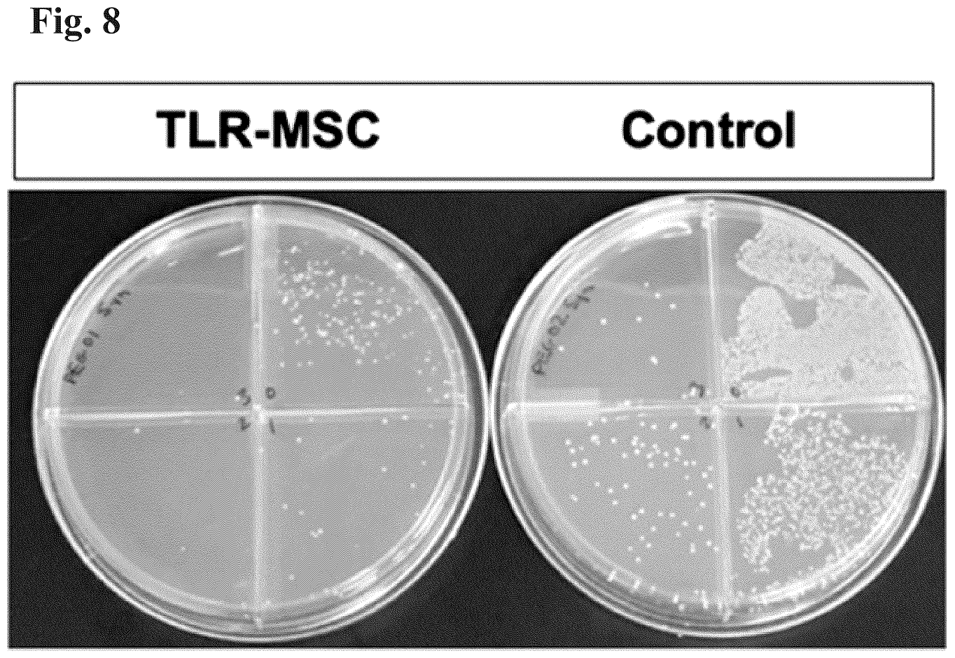

FIG. 8 represents a photographic image of samples from activated MSC treated and untreated joints plated out for bacterial analysis. Synovial tissue bacterial counts. Synovial tissues were collected from the joints of both study horses, and subjected to quantitative culture, as noted above. There were significantly fewer bacterial colonies in the joint tissues from the horse treated with activated MSC plus vancomycin, compared to the horse treated with vancomycin only. Similar results were obtained in a second study in two additional horses.

Table 1 and 2 represent in vitro evaluation of the cytotoxic activity of different conventional antibiotics, as assessed for toxicity against dog cells (Table 1) or horse cells (Table 2). Similar toxicity profiles would also be anticipated for human cells.

The antibiotics with the highest IC50 values are considered non-cytotoxic, while those with lower values are considered more cytotoxic, and unsuitable for co-administration with activated MSC or activated iMSCs. For example, amikacin and enrofloxacin are more cytotoxic to joint cells (e.g. synovial cells and chondrocytes) than vancomycin, and should not be used for preparing the antibiotic plus activated MSCs formulations of uses herein, whereas vancomycin is one of the preferred antibiotics for these formulations for treating gram+ infections.

TABLE 1

| | SYNOVIOCYTES | | | CHONDROCYTES | |
|---|---|---|---|---|---|
| | | IC50 | | | IC50 |
| | Antibiotic | (mg/mL) | | Antibiotic | (mg/mL) |
| 1 | Vancomycin | 13.77 | 1 | Vancomycin | 8.645 |
| 2 | Ampicillin | 3.074 | 2 | Ampicillin | 8.635 |
| | sulbactam | | | sulbactam | |
| 3 | Amikacin | 2.258 | 3 | Ceftazidime | 3.162 |
| 4 | Ceftazidime | 1.622 | 4 | Amikacin | 2.738 |
| 5 | Cefazolin | 1.48 | 5 | Cefazolin | 1.668 |
| 6 | Enrofloxacin | 1.248 | 6 | Enrofloxacin | .078< × <1.56 |

Table 2 represents and overview of equine IC50s of certain antibiotics of use herein

TABLE 2

| | |
|---|---|
| Antibiotic | Concentration mg/ml |

Chondrocytes

| | | |
|---|---|---|
| 1 | Ampicillin Sulbactam | >25 |
| 2 | Imipenem | >25 |
| 3 | Tobramycin | >25 |
| 4 | Amoxicillin | 14.01 |
| 5 | Potassium penicillin | 11.61 |
| 6 | Vancomycin | 7.306 |
| 7 | Enrofloxacin | 4.589 |
| 8 | Ceftiofur Sodium | 4.266 |
| 9 | Cefazolin | 3.948 |
| 10 | Ceftazidime | 3.589 |
| 11 | Florfenicol | 2.19 |
| 12 | Doxycycline | 1.031 |
| 13 | Neomycin | 0.8219 |
| 14 | Gentamicin | 0.7083 |
| 15 | Amikacin | <0.39 |

Synovial Cells

| | | |
|---|---|---|
| 1 | Ampicillin Sulbactam | >25 |
| 2 | Ceftiofur Sodium | >25 |
| 3 | Imipenem | >25 |
| 4 | Amoxicillin | >25 |
| 5 | Potassium penicillin | 15.625< × <31.25 |
| 6 | Tobramycin | 9.49 |
| 7 | Vancomycin | 7.812 |
| 8 | Neomycin | 6.274 |
| 9 | Enrofloxacin | 3.125< × <6.25 |
| 10 | Ceftazidime | 3.359 |
| 11 | Florfenicol | 2.956 |
| 12 | Cefazolin | 1.155 |
| 13 | Amikacin | 0.7993 |
| 14 | Doxycycline | 0.7107 |
| 15 | Gentamicin | 0.5125 |

### MATERIALS AND METHODS

Skeletally mature horses, (male Quarter Horses, 3 years of age), deemed healthy by physical examination, bloodwork (complete blood count, serum biochemical panel), and without evidence of tarsal osteoarthritis determined radiographically were treatment recipients. Two pilot horses were inoculated in one tarsocrural (ankle) joint with methicillin-resistant Staphylococcus aureus (1×10⁶ CFU) and treated on days 1 and 4 with either TLR-activated equine MSC or control lactated ringers' solution. Three healthy, skeletally mature horses donated bone marrow aspirate for mesenchymal stromal cell culture and expansion, which were activated with TLR-3 polyI:C at 10 μg/mL for 2 hours at 2×10⁶ cells/mL in complete growth media immediately prior to IA injection. Both horses received systemic and IA antibiotic therapy (gentamicin 6.6 mg/kg q24 h and potassium penicillin 22,000 IU/kg q6 h IV 10 days and vancomycin 10 mg IA q24 h 7 days). Outcome parameters evaluated included inflammation/pain scoring, serial complete blood count and synovial fluid analyses, quantitative bacterial culture of synovial fluid and synovium at end-term, quantification of cytokines in synovial fluid, imaging (radiographs, ultrasound, MRI), macroscopic scoring at end-term and histology of osteochondral and synovial tissues. Radiographs were assessed and graded (0 absent, 1 present) for soft tissue swelling, articular cartilage erosion, subchondral bone sclerosis, joint space narrowing, irregular joint margins, and osteomyelitis. Gray-scale ultrasound images were assessed and graded (0 normal, 1 mild, 2 moderate, 3 marked) for degree of distention, degree of synovial thickening, degree of fibrinous loculation, degree of vascularity as visualized with power Doppler, as well as character of synovial effusion and presence of hyperechoic foci (0 anechoic/absent, 1 echogenic/present).

All of the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A composition comprising, 1) a Toll like receptor 3 (TLR3) ligand activated or otherwise immune activated mesenchymal stem cells (MSCs) or activated MSC derived from iPSC cells (iMSC); and 2) one or more antimicrobial agent wherein the antimicrobial comprises at least one bactericidal antimicrobial agent with reduced cytotoxicity against mammalian cells.

2. The composition according to claim 1, wherein the at least one antimicrobial agent comprises a bactericidal antibiotic.

3. The composition according to claim 1, wherein the at least one bactericidal antimicrobial agent comprises one or more of a gram-negative bacteria-directed antibiotic, a gram-positive bacteria-directed antibiotic or a combination thereof.

4. The composition according to claim 1, further comprising at least one antimicrobial peptide agent.

5. The composition according to claim 1, wherein the one or more antimicrobial agents comprise one or more of vancomycin, cefazolin, ampicillin-sulbactam, and clindamycin against at least one gram positive bacteria.

6. The composition according to claim 1, wherein the one or more antimicrobial agents comprise one or more of imipenem, ceftriaxone, ceftazidime, and other carbapenem class antibiotics against one or more gram negative bacteria.

7. The composition according to claim 1, wherein the one or more antimicrobial agent does not comprise aminoglycosides, fluoroquinolones, tetracyclines, or neomycin.

8. The composition according to claim 1, wherein the one or more antimicrobial agent does not comprise tetracyclines, macrolides, sulfonamides, lincosamides, trimethoprim, chloramphenicol, or rifampin and further, wherein tetracyclines, macrolides, sulfonamides, lincosamides, trimethoprim, chloramphenicol, and rifampin are excluded from the composition.

9. The composition according to claim 1, wherein the composition comprises formulations for direct delivery to infected joints, tendons, ligaments and associated soft tissues.

10. The composition according to claim 9, wherein the composition comprises a liquid, a gelatinous material or viscous material, a cream, a salve, or a composition on a patch or bandage.

11. The composition according to claim 9, wherein direct delivery comprises delivery to a hip, groin, knee, spine, elbow, wrist, appendages such as hands and feet, ankle, heel, shoulder, neck, or tissues comprising a region having reduced blood supply compared to other regions of a body.

12. The composition according to claim 1, wherein the MSCs comprise autologous or allogeneic MSCs obtained or derived from bone marrow, adipose tissue, cord blood, tissue biopsies, skin biopsies, dental biopsies or other tissues.

13. (canceled)

14. The composition according to claim 1, wherein the TLR3 ligand comprises one or more of polyadenylic polyuridylic acid (poly(A:U), polyinosine polycytidylic acid (pIC), and UV inactivated viral particles.

15. (canceled)

17. The composition according to claim 1, wherein the MSCs or iMSCs comprise human derived MSCs or iMSCs, directly from the subject to be treated or from an unrelated donor.

18. (canceled)

19. A method for treating a subject having a bacterial infection comprising administering a composition according to claim 1 to the subject.

20-24. (canceled)

25. The method according to claim 19, wherein the bacterial infection comprises a bacterial infection of a joint, tendon, ligament, or associated soft tissue infection.

26-27. (canceled)

28. The method according to claim 19, wherein the subject has an acute or chronic bacterial infection of one or more joints, tendons, ligaments, or other associated soft tissues.

29-36. (canceled)

37. A method for treating a bacterial infected joint, tendon, ligament, implant or associated soft tissue in a subject comprising: administering a composition comprising activated MSCs or activated iMSCs and administering at least one antimicrobial agent wherein the antimicrobial comprises at least one bactericidal antimicrobial agent with reduced cytotoxicity against mammalian cells for at least one of at the same time or as a combination composition followed by at least one subsequent composition comprising at least one of activated MSCs or activated iMSCs and at least one bactericidal antimicrobial agent.

38. A pharmaceutical composition comprising a composition according to claim 1; and a pharmaceutically acceptable agent or excipient.

39. (canceled)

40. A kit comprising the composition according to claim 1; and at least one container.

41. (canceled)
